SEQUENCE LISTING (11 sequences)

SEQ ID NO: 1 - DNA, 1308 bp, Artificial Sequence
  synthetic alpha-amylase coding sequence
  CDS (1)...(1308)
atggccaagtacctggagctggaggagggcggcgtgatcatgcaggcgttctactgggacgtcccgagcggaggcatctggtgggacaccatccgccagaagatccccgagtggtacgacgccggcatctccgcgatctggataccgccagcttccaagggcatgtccgggggctactcgatgggctacgacccgtacgactacttcgacctcggcgagtactaccagaagggcacggtggagacgcgcttcgggtccaagcaggagctcatcaacatgatcaacacggcgcacgcctacggcatcaaggtcatcgcggacatcgtgatcaaccacagggccggcggcgacctggagtggaacccgttcgtcggcgactacacctggacggacttctccaaggtcgcctccggcaagtacaccgccaactacctcgacttccaccccaacgagctgcacgcgggcgactccggcacgttcggcggctacccggacatctgccacgacaagtcctgggaccagtactggctctgggcctcgcaggagtcctacgcggcctacctgcgctccatcggcatcgacgcgtggcgcttcgactacgtcaagggctacggggcctgggtggtcaaggactggctcaactggtggggcggctgggcggtgggcgagtactgggacaccaacgtcgacgcgctgctcaactgggcctactcctccggcgccaaggtgttcgacttccccctgtactacaagatggacgcggccttcgacaacaagaacatcccggcgctcgtcgaggccctgaagaacggcggcacggtggtctcccgcgacccgttcaaggccgtgaccttcgtcgccaaccacgacacggacatcatctggaacaagtacccggcgtacgccttcatcctcacctacgagggccagcccacgatcttctaccgcgactacgaggagtggctgaacaaggacaagctcaagaacctgatctggattcacgacaacctcgcgggcggctccactagtatcgtgtactacgactccgacgagatgatcttcgtccgcaacggctacggctccaagcccggcctgatcacgtacatcaacctgggctcctccaaggtgggccgctgggtgtacgtcccgaagttcgccggcgcgtgcatccacgagtacaccggcaacctcggcggctgggtggacaagtacgtgtactcctccggctgggtctacctggaggccccggcctacgaccccgccaacggccagtacggctactccgtgtggtcctactgcggcgtcggc

SEQ ID NO: 2 - protein, 436 aa, Artificial Sequence
  SYNAMYLASE
MAKYLELEEGGVIMQAFYWDVPSGGIWWDTIRQKIPEWYDAGISAIWIPPASKGMSGGYSMGYDPYDYFDLGEYYQKGTVETRFGSKQELINMINTAHAYGIKVIADIVINHRAGGDLEWNPFVGDYTWTDFSKVASGKYTANYLDFHPNELHAGDSGTFGGYPDICHDKSWDQYWLWASQESYAAYLRSIGIDAWRFDYVKGYGAWVVKDWLNWWGGWAVGEYWDTNVDALLNWAYSSGAKVFDFPLYYKMDAAFDNKNIPALVEALKNGGTVVSRDPFKAVTFVANHDTDIIWNKYPAYAFILTYEGQPTIFYRDYEEWLNKDKLKNLIWIHDNLAGGSTSIVYYDSDEMIFVRNGYGSKPGLITYINLGSSKVGRWVYVPKFAGACIHEYTGNLGGWVDKYVYSSGWVYLEAPAYDPANGQYGYSVWSYCGVG

SEQ ID NO: 3 - DNA, 22 bp, Artificial Sequence
  oligonucleotide primer - synamylase
caagcaggagctcatcaacatg

SEQ ID NO: 4 - DNA, 20 bp, Artificial Sequence
  oligonucleotide primer - synamylase
gccctgtggttgatcacgat

SEQ ID NO: 5 - DNA, 23 bp, Artificial Sequence
  oligonucleotide probe - synamylase
tccgcgatgaccttgatgccgta

SEQ ID NO: 6 - DNA, 21 bp, Artificial Sequence
  oligonucleotide primer - EC gene
gaacgtgtgttgggtttgcat

SEQ ID NO: 7 - DNA, 20 bp, Artificial Sequence
  oligonucleotide primer - EC gene
tccagcaatccttgcacctt

SEQ ID NO: 8 - DNA, 24 bp, Artificial Sequence
  oligonucleotide probe - EC gene
tgcagcctaaccatgcgcagggta

SEQ ID NO: 9 - DNA, 18 bp, Artificial Sequence
  oligonucleotide primer - PMI
ccgggtgaatcagcgttt

SEQ ID NO: 10 - DNA, 18 bp, Artificial Sequence
  oligonucleotide primer - PMI
gccgtggcctttgacagt

SEQ ID NO: 11 - DNA, 20 bp, Artificial Sequence
  oligonucleotide probe - PMI
tgccgccaacgaatcaccgg

(19) United States
(12) Patent Application Publication
(10) Pub. No.: US 2007/0269820 A1
(43) Pub. Date: Nov. 22, 2007

(54) Methods for the detection of nucleic acid encoding ALPHA-amylase

(75) Inventor: Mary Fielder, Holly Springs, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Syngenta Participations AG, Basel (CH)

(21) Appl. No.: 11/639,560

(22) Filed: Dec. 15, 2006

Related U.S. Application Data
(60) Provisional application No. 60/751,247, filed on Dec. 16, 2005.

(51) Int. Cl. C12Q 1/68

(52) U.S. Cl. 435/6

(57) ABSTRACT

The present invention is drawn to methods and compositions for the detection and/or quantification of nucleic acid encoding α-amylase in a sample. Provided herein are novel primer and probe compositions for use in detecting the presence of α-amylase in an organism, particularly using quantitative PCR methods. Provided herein are novel oligonucleotide primers and probes, including the primers set forth in SEQ ID NO:3 and 4 and the novel oligonucleotide probe sequence set forth in SEQ ID NO:5, including variants, fragments and complements thereof, kits comprising these primer and probe sequences, and methods for using these primers and probes for the detection and/or quantification of α-amylase in an organism, particularly a genetically-modified organism.

### CROSS REFERENCE TO RELATED APPLICATION

This application claims the benefit of U.S. Provisional Application Ser. No. 60/751,247, filed Dec. 16, 2005, the contents of which are herein incorporated by reference in their entirety.

### FIELD OF THE INVENTION

This invention provides compositions and methods for the detection and/or quantification of a nucleic acid encoding ALPHA-amylase in an organism, particularly using the polymerase chain reaction.

### BACKGROUND OF THE INVENTION

Endo-1,4-α-D-glucan glucohydrolase (α-amylase, EC 3.2.1.1) is currently used in a broad array of industrial applications. These include starch hydrolysis for the production of ethanol and high fructose corn syrup, starch soil removal in laundry washing powders and dish-washing detergents, textile de-sizing, the production of modified starches, baking, hydrolysis of oil-field drilling fluids, and paper recycling.

Corn is milled to obtain cornstarch and other corn-milling co-products such as corn gluten feed, corn gluten meal, and corn oil. The starch obtained from the process is often further processed into other products such as derivatized starches and sugars, or fermented to make a variety of products including alcohols or lactic acid. Processing of cornstarch often involves the use of enzymes, in particular, enzymes that hydrolyze and convert starch into fermentable sugars or fructose (e.g., α-amylase).

Recently a group of thermostable α-amylase genes from nature were identified (Richardson et al. (2002) J. Biol. Chem. 277 (29):26501-26507) and subsequent laboratory evolution of novel and improved chimeric α-amylase enzymes with performance characteristics ideal for the corn wet milling process have been developed. Additionally, transgenic plants have been developed in which a thermostable α-amylase enzyme is introduced into the plants. These plants perform well in fermentation without the addition of exogenous α-amylase, require much less time for liquefaction, and result in more complete solubilization of starch (U.S. Pat. No. 7,102,057).

Current methods for the detection of α-amylase activity in an organism involve the calorimetric Amylazyme Assay (Megazyme, Bray Co., Wicklow, Ireland). Additional methods that measure the presence of the nucleic acid encoding the α-amylase enzyme are needed.

### SUMMARY OF THE INVENTION

The present invention is drawn to methods and compositions for the detection of a nucleic acid encoding ALPHA-amylase (“α-amylase”) in an organism or test sample derived from an organism, particularly from a genetically modified plant or microorganism. Provided herein are novel primer and probe compositions for use in detecting the presence of a nucleic acid encoding α-amylase in an organism, particularly using quantitative PCR methods.

In one embodiment, the present invention provides novel oligonucleotide primers and probe sets. These primers and probe sets can be used in amplification methods (such as PCR, particularly TAQMAN®-based PCR) and packaged into kits for use in amplification methods for the purpose of detecting a nucleic acid encoding α-amylase in an organism. Additionally, these primers and/or probe sets can be used in amplification methods (such as polymerase chain reaction) to evaluate the expression level of endogenous or heterologous α-amylase and/or to confirm integration of a heterologous nucleic acid encoding α-amylase in an organism in which the nucleic acid encoding α-amylase has been introduced.

Thus, in one embodiment, the present invention provides for novel oligonucleotide primers comprising SEQ ID NO:3 and 4, as well as variants, fragments and complements thereof, and the novel oligonucleotide probe sequence set forth in SEQ ID NO:5, as well as variants, fragments, and complements thereof. Further provided are kits useful for the detection and/or quantification of nucleic acid encoding α-amylase in a sample comprising a composition according to the present invention. The kits may further comprise instructions for using the provided composition in a polymerase-based amplification reaction, e.g., PCR or QPCR.

In another embodiment, the present invention relates to a method of detecting a nucleic acid encoding α-amylase in a sample using polymerase-based amplification of a target nucleic acid region present in the organism, the method comprising: (a) providing a test sample suspected of containing nucleic acid encoding α-amylase, (b) contacting the sample with a composition of the invention under conditions sufficient to provide polymerase-based nucleic acid amplification products comprising the target nucleic acid region; and (c) detecting the presence of the nucleic acid amplification product as an indication of the presence of nucleic acid encoding α-amylase in the test sample.

### DETAILED DESCRIPTION

Overview

The methods and compositions of the present invention are directed to the detection and/or quantification of a nucleic acid encoding α-amylase in an organism or a test sample derived from an organism, particularly using a polymerase-based amplification method. As used herein, “polymerase chain reaction” or “PCR” refers to an in vitro method for amplifying a specific polynucleotide template sequence (or “target nucleic acid”). The target nucleic acid encoding α-amylase that is detectable using the compositions and methods of the present invention is provided in U.S. Pat. No. 7,102,057, herein incorporated by reference in its entirety, and in SEQ ID NO:1. The methods and compositions are useful in the detection and/or quantification of a nucleic acid encoding α-amylase in various microorganisms, including but not limited to Escherichia coli, Schizosaccharomyces pombe, and Pichia pastoris, as well as in plants, including but not limited to maize, wheat, rice, canola, and alfalfa. In particular, the compositions and methods are useful for the detection and/or quantification of a nucleic acid encoding α-amylase in genetically-modified organisms in which the nucleic acid encoding α-amylase has been introduced. A “genetically-modified organism” refers to an organism that has incorporated or integrated heterologous nucleic acid sequences or DNA fragments into at least one cell of the organism. “Heterologous” generally refers to the nucleic acid sequences that are not endogenous to the cell or part of the native genome in which they are present, and have been added to the cell by infection, transfection, microinjection, electroporation, microprojection, or the like.

Nucleic acid material can be extracted from an organism of interest using routine techniques known in the art. “Nucleic acid extracted from an organism of interest” is understood as meaning either the total nucleic acid, or the ribosomal RNA or the genomic DNA derived from the organism, or even the nucleic acid obtained from the reverse transcription of nucleic acid from the organism. Nucleic acid material is extracted using standard methods known in the art (see, for example, Ausubel et al. (1994) Current Protocols in Molecular Biology, John Wiley & Sons, New York, N.Y.). Additionally, commercially available kits can be employed in the present methods using the manufacturer's instructions.

Oligonucleotide Primers

In one embodiment of the present invention, oligonucleotide primers are provided for use in the detection and/or quantification of nucleic acid encoding α-amylase in a sample. As used herein, a “primer” refers to a type of oligonucleotide having or containing a sequence complementary to a target polynucleotide present in or derived from an organism comprising a nucleic acid encoding α-amylase, which hybridizes to the target polynucleotide through base pairing. In one embodiment, the primers of the invention are those comprising the nucleotide sequences set forth in SEQ ID NO:3 and 4, as well as variants, fragments, and complements thereof. The term “oligonucleotide” refers to a short polynucleotide sequence, typically less than or equal to 100 nucleotides long (e.g., between about 5 and about 100 nucleotides, preferably between about 7 to about 75 nucleotides, more preferably between 10 to 25 nucleotides in length). However, the term is also intended to encompass longer or shorter polynucleotide chains.

As used herein, the terms “target polynucleotide” and “target nucleic acid” refer to a polynucleotide whose presence and/or amount is to be determined in a sample. In the present invention, the target nucleic acid corresponds to the nucleic acid that encodes all or a part of an α-amylase enzyme. A “target nucleic acid” of the present invention contains a known sequence of at least about 20 nucleotides, preferably at least 50 nucleotides, more preferably at least about 100 or more nucleotides, for example, 500 or more nucleotides. A “target nucleic acid” of the invention may be a naturally occurring polynucleotide (i.e., one existing in nature without human intervention), or a recombinant polynucleotide (i.e., one existing only with human intervention), including but not limited to genomic DNA, cDNA, plasmid DNA, total RNA, mRNA, tRNA, and rRNA. The target nucleic acid also includes amplified products of itself, for example, as in a polymerase chain reaction. According to the invention, a “target polynucleotide” or “target nucleic acid” may contain one or more modified nucleotides, including, for example, phosphorothioate, phosphite, ring atom modified derivatives, and the like. In some embodiments, the target nucleic acid corresponds to nucleotides 258-341 of SEQ ID NO:1.

As used herein, the term “complementary” refers to the concept of sequence complementarity between regions of two polynucleotide strands or between two regions of the same polynucleotide strand. A first region of a polynucleotide is complementary to a second region of the same or a different polynucleotide if, when the two regions are arranged in an antiparallel fashion, at least one nucleotide of the first region is capable of base pairing with a base of the second region. Therefore, it is not required for two complementary polynucleotides to base pair at every nucleotide position. “Complementary” refers to a first polynucleotide that is 100% or “fully” complementary to a second polynucleotide and thus forms a base pair at every nucleotide position. “Complementary” also refers to a first polynucleotide that is not 100% complementary (e.g., 90%, or 80% or 70% complementary) and contains mismatched nucleotides at one or more nucleotide positions. Thus, the oligonucleotides of the present invention are capable of detecting nucleic acids encoding α-amylase that differ in the target nucleic acid region, so long as the target nucleic acid region is capable of hybridizing to the polynucleotides disclosed herein as SEQ ID NO:3, 4, or 5.

As used herein, the term “hybridization” is used in reference to the pairing of complementary (including partially complementary as discussed above) polynucleotide strands. Hybridization and the strength of hybridization (i.e., the strength of the association between polynucleotide strands) is impacted by many factors well known in the art including the degree of complementarity between the polynucleotides; stringency of the conditions involved that are affected by such conditions as the concentration of salts; the melting temperature (Tm) of the formed hybrid; the presence of other components; the molarity of the hybridizing strands; and, the G:C content of the polynucleotide strands.

As used herein, “Tm” and “melting temperature” are interchangeable terms which are the temperature at which 50% of a population of double-stranded polynucleotide molecules becomes dissociated into single strands. The equation for calculating the Tm of polynucleotides is well known in the art. For example, the Tm may be calculated by the following equation: Tm=69.3+0.41×(G+C) %−650/L, wherein L is the length of the probe in nucleotides. The Tm of a hybrid polynucleotide may also be estimated using a formula adopted from hybridization assays in 1 M salt, and commonly used for calculating Tm for PCR primers: [(number of A+T)×2° C.+(number of G+C)×4° C.], see, for example, Newton et al. (1997) PCR 2nd Ed. (Springer-Verlag, New York). Other more sophisticated computations exist in the art, which take structural as well as sequence characteristics into account for the calculation of Tm. A calculated Tm is merely an estimate; the optimum temperature is commonly determined empirically.

The primers of the present invention can be prepared using techniques known in the art, including, but not limited to, cloning and digestion of the appropriate sequences and direct chemical synthesis.

Chemical synthesis methods that can be used to make the primers of the present invention, include, but are not limited to, the phosphotriester method described by Narang et al., Methods in Enzymology, 68:90 (1979), the phosphodiester method disclosed by Brown et al., Methods in Enzymology, 68:109 (1979), the diethylphosphoramidate method disclosed by Beaucage et al., Tetrahedron Letters, 22:1859 (1981) and the solid support method described in U.S. Pat. No. 4,458,066. The use of an automated oligonucleotide synthesizer to prepare synthetic oligonucleotide primers of the present invention is also contemplated herein. Additionally, if desired, the primers can be labeled using techniques known in the art and described below.

Oligonucleotide Probes

In some embodiments of the present invention, the oligonucleotide primers disclosed herein can be used in a PCR reaction for the detection of nucleic acid encoding α-amylase. In a preferred embodiment, one or more of the oligonucleotide primers of the present invention may be used in combination with one or more probe sequences. Preferably, the probes are separate from the oligonucleotide primers (“bimolecular probes,” including, for example TAQMAN® probes or Molecular Beacon probes); however, it is contemplated that the probes can be attached to one of the oligonucleotide primers (“unimolecular probes” or “tailed probes,” including, for example SCORPIONS™ primers and AMPLIFLUOR® Direct Primers). See, for example, Holland et al. (1991) Proceedings of the National Academy of Science (USA) 88(16): 7276-7280; Lee et al. (1993) Nucleic Acids Research 21(16): 3761-3766; Tyagi and Kramer (1996) Nature Biotechnology 14(3):303-308; Nazarenko et al. (1997) Nucleic Acids Research 25(12): 2516-2521; U.S. Pat. Nos. 5,866,366; 6,090,592; 6,117,635; and 6,117,986).

As used herein, the term “probe” refers to a polynucleotide that forms a hybrid structure with the denatured target nucleic acid in a region of the target nucleic acid that is between the two regions that are complementary to the oligonucleotide primers. In some embodiments, the probe can be complementary to a primer extension product due to complementarity of at least one sequence in the probe with a sequence in the primer extension product. By “primer extension product” is intended the nucleic acid product that results from polymerase-based extension (using the target nucleic acid as a template) of the oligonucleotide primer comprising the sequences disclosed herein as SEQ ID NO:3 and 4, as well as variants, fragments, and complements thereof. The polynucleotide regions of the probe can be composed of DNA and/or RNA and/or synthetic nucleotide analogs. Preferably, the probe does not contain a sequence complementary to the oligonucleotide primer sequence(s) described above, nor does it bind a region of the target nucleic acid that is complementary to the oligonucleotide primer sequences. The probe of the present invention is ideally less than or equal to 100 nucleotides in length, for example less than or equal to 80, 70, 60, 50, 40, 30, 20, or less than 10 nucleotides in length.

As is understood in the art, the oligonucleotide primers and/or probes set forth in SEQ ID NO:3-5 may further comprise additional sequences or moieties to facilitate hybridization to the target nucleic acid or primer extension product, to facilitate attachment of a quencher, fluorophore, PCR blocker or other suitable moiety, or to facilitate the formation of a desired secondary structure (e.g., attachment of additional nucleotides at the 3′ and/or 5′ terminus of the oligonucleotide primer or probe sequences for the formation of a stem loop structure as known in the art). In one embodiment, the probe sequence further comprises a phosphate moiety at the 3′ terminus to prevent polymerase-based extension of the oligonucleotide probe sequence.

It is further understood that variants and fragments of the oligonucleotide primer and/or probe sequences disclosed herein can be used in the methods of the invention. For example, the sequences can be shorter or longer than the sequences disclosed herein as SEQ ID NO:3-5, or may have 1 to 5, or 5 to 10, nucleotide substitutions so long as the oligonucleotide primers retain the ability to hybridize to the target nucleic acid in such a manner as to initiate (under the appropriate conditions as described elsewhere herein) the template-dependent extension of the primer sequence in a PCR or equivalent reaction, and so long as the probe retains the ability to hybridize to the target nucleic acid under the appropriate conditions.

Labeling

The primers and/or probes of the present invention can further include one or more labels to facilitate monitoring of amplification reactions. As used herein, the term “label” or “labeled” refers to any atom or moiety which can be used to provide a detectable (preferably, quantifiable) signal, and which can be attached to a polynucleotide, oligonucleotide primer or probe. A wide variety of labels and conjugation techniques, including direct and indirect labeling, are known and are reported extensively in both the scientific and patent literature. Examples of labels that can be used include radionucleotides, enzymes, substrates, cofactors, inhibitors, fluorescent moieties, intercalators, chemiluminescent moieties, magnetic particles, and the like. Patents teaching the use of such labels include U.S. Pat. Nos. 3,817,837; 3,850,752; 3,939,350; 3,996,345; 4,277,437; 4,275,149; and 4,366,241.

Fluorophore and Quencher

In one embodiment, the probes can be labeled with a fluorophore and a quencher in such a manner that the fluorescence emitted by the fluorophore in probes that are bound to target nucleic acid is substantially quenched, whereas the fluorescence in probes that are not bound to target nucleic acid is not quenched, resulting in an increase in overall fluorescence upon cleavage of the probe by the polymerase-based extension of the oligonucleotide primer sequence(s). The generation of a fluorescent signal during real-time detection of the amplification products allows accurate quantification of the initial number of target sequences in a sample.

A wide variety of fluorophores can be used, including but not limited to: 5-FAM (also called 5-carboxyfluorescein; also called Spiro(isobenzofuran-1(3H), 9′-(9H)xanthene)-5-carboxylic acid, 3′,6′-dihydroxy-3-oxo-6-carboxyfluorescein); 5-Hexachloro-Fluorescein ([4,7,2′,4′,5′,7′-hexachloro-(3′,6′-dipivaloyl-fluoresceinyl)-6-carboxylic acid]); 6-Hexachloro-Fluorescein ([4,7,2′,4′,5′,7′-hexachloro-(3′,6′-dipivaloylfluoresceinyl)-5-carboxylic acid]); 5-Tetrachloro-Fluorescein ([4,7,2′,7′-tetrachloro-(3′,6′-dipivaloylfluoresceinyl)-5-carboxylic acid]); 6-Tetrachloro-Fluorescein ([4,7,2′,7′-tetrachloro-(3′,6′-dipivaloylfluoresceinyl)-6-carboxylic acid]); Xanthylium, 9-(2,4-dicarboxyphenyl)-3,6-bis(dimethylamino); 6-TAMRA (6-carboxytetramethylrhodamine; Xanthylium, 9-(2,5-dicarboxyphenyl)-3,6-bis(dimethylamino); EDANS (5-((2-aminoethyl)amino)naphthalene-1-sulfonic acid); 1,5-IAEDANS (5-((((2-iodoacetyl)amino)ethyl)amino)naphthalene-1-sulfonic acid); DABCYL (4-((4-(dimethylamino)phenyl)azo)benzoic acid) Cy5 (Indodicarbocyanine-5) Cy3 (Indo-dicarbocyanine-3); and BODIPY FL (2,6-dibromo-4,4-difluoro-5,7-dimethyl-4-bora-3a,4a-diaza-s-indacene-3-proprionic acid), Rox, as well as suitable derivatives thereof.

As used herein, the term “quencher” refers to a chromophoric molecule or part of a compound, which is capable of reducing the emission from a fluorescent donor when attached to or is in proximity to the donor. Quenching may occur by any of several mechanisms including fluorescence resonance energy transfer (FRET), photoinduced electron transfer, paramagnetic enhancement of intersystem crossing, Dexter exchange coupling, and exciton coupling such as the formation of dark complexes. Therefore, the quencher can be any material that can quench at least one fluorescence emission from an excited fluorophore being used in the assay. There is a great deal of practical guidance available in the literature for selecting appropriate reporter-quencher pairs for particular probes, as exemplified by the following references: Clegg (1993) Proc. Natl. Acad. Sci. USA 90:2994-2998; Wu et al. (1994) Anal. Biochem. 218:1-13; Pesce et al., editors (1971) Fluorescence Spectroscopy (Marcel Dekker, New York); White et al. (1970) Fluorescence Analysis. A Practical Approach (Marcel Dekker, New York); and the like. The literature also includes references providing exhaustive lists of fluorescent and chromogenic molecules and their relevant optical properties for choosing reporter-quencher pairs, e.g., Berlman (1971) Handbook of Fluorescence Spectra of Aromatic Molecules, 2nd Edition (Academic Press, New York); Griffiths (1976) Colour and Constitution of Organic Molecules (Academic Press, New York); Bishop, editor (1972) Indicators (Pergamon Press, Oxford); Haugland (1992) Handbook of Fluorescent Probes and Research Chemicals (Molecular Probes, Eugene, Oreg.); Pringsheim (1949) Fluorescence and Phosphorescence (Interscience Publishers, New York), all of which incorporated herein by reference in their entirety. Further, there is extensive guidance in the literature for derivatizing reporter and quencher molecules for covalent attachment via common reactive groups that can be added to an oligonucleotide, as exemplified by the following references, see, for example, Hauglans, 1992, supra; Ullman et al., U.S. Pat. No. 3,996,345; Khanna et al., U.S. Pat. No. 4,351,760, all of which herein incorporated by reference.

A number of commercially available quenchers are known in the art, and include but are not limited to DABCYL, 5-TAMRA (5-carboxytetramethylrhodamine); and black hole quenchers (Biosearch Technologies, Inc., Novato, Calif.).

Attachment of Fluorophore and Quencher

The probe according to the present invention preferably has both a fluorophore and a quencher attached to the probe sequence. Attachment of the quencher is preferably at the 3′ terminal nucleotide, and attachment of the fluorophore is preferably at the 5′ terminal nucleotide of the probe, although the orientation of the fluorophore and quencher can vary. In another embodiment, one or the other of the fluorophore or quencher can be attached anywhere within the probe, preferably at a distance from the other of the fluorophore/quencher such that sufficient amount of quenching occurs when the oligonucleotide probe is bound to the target nucleic acid.

Attachment can be made via direct coupling, or alternatively using a linker sequence or other suitable molecule of between 1 and 5 atoms in length. Linkage can be made using any of the means known in the art. Appropriate linking methodologies for attachment of many dyes to oligonucleotides are described in many references, e.g., Marshall (1975) Histochemical J. 7: 299-303; Menchen et al., U.S. Pat. No. 5,188,934; Menchen et al., European Patent Application 87310256.0; and Bergot et al., International Application PCT/US90/05565, each of which is herein incorporated by reference.

Attachment of Probes to a Solid Support

The probes of the present invention may also be linked to a solid support either directly, or through a chemical spacer. A solid support useful according to the invention includes but is not limited to silica-based matrices, cellulosic materials, plastic materials, membrane-based matrices and beads comprising surfaces including, but not limited to styrene, latex or silica based materials and other polymers. Magnetic beads are also useful according to the invention. Solid supports can be obtained commercially from several manufacturers.

It is well known by those with skill in the art that oligonucleotides can be synthesized with certain chemical and/or capture moieties, such that they can be coupled to solid supports. Examples of attaching oligonucleotides to solid supports can be found, for example, in U.S. Patent Application No. U.S. 2003/0165912 A1, which is herein incorporated by reference in its entirety. Suitable capture moieties include, but are not limited to, biotin, a hapten, a protein, a nucleotide sequence, an antigenic moiety, or a chemically reactive moiety. Such oligonucleotides may either be used first in solution and then captured onto a solid support, or first attached to a solid support and then used in a detection reaction.

Polymerase-Based Amplification

Numerous different PCR or QPCR protocols are known in the art and can be directly applied or adapted for use using the presently-described compositions for the detection and/or quantification of nucleic acid encoding α-amylase in a sample. Generally, in PCR, a target polynucleotide sequence is amplified by reaction with at least one oligonucleotide primer or pair of oligonucleotide primers. The primers hybridize to a complementary region of the target nucleic acid and a DNA polymerase extends the primer(s) to amplify the target sequence. Under conditions sufficient to provide polymerase-based nucleic acid amplification products, a nucleic acid fragment of one size dominates the reaction products (the target polynucleotide sequence which is the amplification product). The amplification cycle is repeated to increase the concentration of the single target polynucleotide sequence. The reaction can be performed in any thermocycler commonly used for PCR. However, preferred are cyclers with real-time fluorescence measurement capabilities, for example, ABI 7900HT® (Applied Biosystems, Foster City, Calif.), ROTOR-GENE™ (Corbett Research, Sydney, Australia), LIGHTCYCLER® (Roche Diagnostics Corp, Indianapolis, Ind.), ICYCLER® (Biorad Laboratories, Hercules, Calif.), SMARTCYCLER® (Cepheid, Sunnyvale, Calif.), and MX4000® (Stratagene, La Jolla, Calif.).

Quantitative PCR (QPCR) (also referred as real-time PCR) is preferred under some circumstances because it provides not only a quantitative measurement, but also reduced time and contamination. As used herein, “quantitative PCR (or “real time QPCR”) refers to the direct monitoring of the progress of a PCR amplification as it is occurring without the need for repeated sampling of the reaction products. In quantitative PCR, the reaction products may be monitored via a signaling mechanism (e.g., fluorescence) as they are generated and are tracked after the signal rises above a background level but before the reaction reaches a plateau. The number of cycles required to achieve a detectable or “threshold” level of fluorescence varies directly with the concentration of amplifiable targets at the beginning of the PCR process, enabling a measure of signal intensity to provide a measure of the amount of target nucleic acid in a sample in real time.

In a preferred embodiment, a labeled probe is used to detect the extension product generated by PCR amplification. Any probe format utilizing a labeled probe comprising the sequences of the invention may be used, e.g., such as TAQMAN® probes, SCORPIONS™ probes, AMPLIFLUOR® Direct Primers, or molecular beacon probes as is known in the art. It will be readily apparent to one of ordinary skill in the art that appropriate modifications to the primers and probes disclosed herein will be necessary to utilize various primer/probe formats, as discussed supra.

PCR Conditions

Methods for setting up a PCR reaction are well known to those skilled in the art. The reaction mixture minimally comprises template nucleic acid (except in the case of a negative control as described below) and oligonucleotide primers and/or probes in combination with suitable buffers, salts, and the like, and an appropriate concentration of a nucleic acid polymerase. As used herein, “nucleic acid polymerase” refers to an enzyme that catalyzes the polymerization of nucleoside triphosphates. Generally, the enzyme will initiate synthesis at the 3′-end of the primer annealed to the target sequence, and will proceed in the 5′-direction along the template until synthesis terminates. An appropriate concentration includes one which catalyzes this reaction in the presently described methods. Known DNA polymerases include, for example, E. coli DNA polymerase I, T7 DNA polymerase, Thermus thermophilus (Tth) DNA polymerase, Bacillus stearothermophilus DNA polymerase, Thermococcus litoralis DNA polymerase, Thermus aquaticus (Taq) DNA polymerase and Pyrococcus furiosus (Pfu) DNA polymerase. In a preferred embodiment, the DNA polymerase has 5′→3′ exonuclease activity. The reaction may further include an antibody specific to the DNA polymerase, wherein binding of the antibody blocks activity of the polymerase. This component is particularly preferred in a hot-start PCR method where activation of the polymerase results from the temperature-dependent dissociation of the antibody from the polymerase.

In addition to the above components, the reaction mixture produced in the subject methods includes primers, probes and deoxyribonucleoside triphosphates (dNTPs). The primers are present at about 10 to about 1500 nM, about 50 to about 1200 nM, about 100 to about 1000 nM, or about 250 nM. The probe is present at about 10 to about 500 nM, or about 50 to about 400 nM, or about 75 to about 300 nM, or about 100 nM.

Usually the reaction mixture will further comprise four different types of dNTPs corresponding to the four-naturally occurring nucleoside bases, i.e. dATP, dTTP, dCTP and dGTP. In the subject methods, each dNTP will typically be present at a final concentration in the reaction mixture ranging from about 10 to 5000 μM, usually from about 20 to 1000 μM, about 100 to 800 μM, about 300 to 600 μM, or about 200 μM.

The reaction mixture prepared in the first step of the subject methods further includes an aqueous buffer medium that includes a source of monovalent ions, a source of divalent cations and a buffering agent. Any convenient source of monovalent ions, such as potassium chloride, potassium acetate, ammonium acetate, potassium glutamate, ammonium chloride, ammonium sulfate, and the like may be employed. The divalent cation may be magnesium, manganese, zinc and the like, where the cation will typically be magnesium. Any convenient source of magnesium cation may be employed, including magnesium chloride, magnesium acetate, and the like. The amount of magnesium present in the buffer may range from 0.5 to 10 mM, but will preferably range from about 1 to about 6 mM, about 3 to about 5 mM, or about 1.5 mM. Representative buffering agents or salts that may be present in the buffer include Tris, Tricine, HEPES, MOPS and the like, where the amount of buffering agent will typically range from about 5 to 150 mM, usually from about 7 to 100 mM, and more usually from about 10 to 50 mM, where in certain preferred embodiments the buffering agent will be present in an amount sufficient to provide a pH ranging from about 6.0 to 9.5, or about pH 8.3. Other agents which may be present in the buffer medium include stabilizers and/or chelating agents such as EDTA, EGTA and the like.

The primers can be used in the reaction at a final concentration of at least about 10 nM to about 2 uM, about 100 nM to about 1 uM, about 500 nM to about 900 nM, or about 900 nM. The probe can be used in the reaction at a final concentration of at least about 10 nM to about 2 uM, about 50 nM to about 1 uM, or about 100 nM. One of skill in the art will recognize that the primer and/or probe concentrations can be optimized for the particular application and/or source of target nucleic acid.

In preparing the reaction mixture, the various constituent components may be combined in any convenient order. For example, the buffer may be combined with primer, polymerase, and then template nucleic acid, or all of the various constituent components may be combined at the same time to produce the reaction mixture.

Alternatively, commercially available premixed reagents can be utilized in the methods of the invention according to the manufacturer's instructions, or modified to improve reaction conditions (e.g., modification of buffer concentration, cation concentration, or dNTP concentration, as necessary), including, for example, TAQMAN® Universal PCR Master Mix (Applied Biosystems), JUMPSTART™ Taq READYMIX® (Sigma-Aldrich, St. Louis, Mo.), OMNIMIX® or SMARTMIX® (Cepheid), IQ™ Supermix (Bio-Rad Laboratories), LIGHTCYCLER® FastStart (Roche Applied Science, Indianapolis, Ind.), or BRILLIANT® QPCR Master Mix (Stratagene, La Jolla, Calif.).

Following preparation of the reaction mixture, the reaction mixture is subjected to primer extension reaction conditions (“conditions sufficient to provide polymerase-based nucleic acid amplification products”), i.e., conditions that permit for polymerase mediated primer extension by addition of nucleotides to the end of the primer molecule using the template strand as a template. In many embodiments, the primer extension reaction conditions are amplification conditions, which include a plurality of reaction cycles, where each reaction cycle comprises: (1) a denaturation step, (2) an annealing step, and (3) a polymerization (i.e., “extension”) step. The number of reaction cycles will vary depending on the application being utilized, but will usually be at least about 15, at least about 20, or at least about 60 cycles or higher, where the number of cycles will typically range from about 20 to 40. For methods where more than about 25, usually more than about 30 cycles are performed, it may be convenient or desirable to introduce additional polymerase into the reaction mixture such that conditions suitable for enzymatic primer extension are maintained. Prior to cycling, the reaction conditions may comprise an initial denaturation/polymerase activation step at about 95° C. for about 1, about 2, about 3, about 4, or about 5 or more minutes. Following cycling, the reaction conditions may comprise a final extension step at about 60° C. to about 80° C., or about 72° C. for about 0.5, about 1, about 2, about 3, about 4, or about 5 or more minutes, although this step is usually only necessary when performing standard PCR (i.e., non-real time PCR) in which the PCR products will be evaluated at the completion of the cycling reactions.

The denaturation step comprises heating the reaction mixture to an elevated temperature and maintaining the mixture at the elevated temperature for a period of time sufficient for any double stranded or hybridized nucleic acid present in the reaction mixture to dissociate. For denaturation, the temperature of the reaction mixture will usually be raised to, and maintained at, a temperature ranging from about 85° C. to 100° C., usually from about 90° C. to 98° C. and more usually from about 93° C. to 96° C., or about 95° C. for a period of time ranging from about 3 to 120 seconds, usually from about 5 to about 30 seconds, or about 10 to about 15 seconds.

Following denaturation, the reaction mixture will be subjected to conditions sufficient for primer annealing to template nucleic acid present in the mixture (if present), and for polymerization of nucleotides to the primer ends in a manner such that the primer is extended using the nucleic acid to which it is hybridized as a template, i.e., conditions sufficient for enzymatic production of primer extension product. The temperature to which the reaction mixture is lowered to achieve these conditions will usually be chosen to provide optimal efficiency and specificity, and will generally range from about 50° C. to 75° C., usually from about 55° C. to 70° C. and more usually from about 60° C. to 68° C., more particularly around 60° C. This temperature is often determined based on the Tm of the primer sequences, which can be calculated as discussed above. Annealing/extension conditions will be maintained for a period of time ranging from about 15 seconds to 30 min, usually from about 20 seconds to about 5 minutes, or about 30 seconds to about 1 minute, or about 60 seconds.

This step can optionally comprise one of each of an annealing step and an extension step with variation and optimization of the temperature and length of time for each step. In a 2-step annealing and extension, the annealing step is allowed to proceed as above. Following annealing of primer to template nucleic acid, the reaction mixture will be further subjected to conditions sufficient to provide for polymerization of nucleotides to the primer ends as above. To achieve polymerization conditions, the temperature of the reaction mixture will typically be raised to or maintained at a temperature ranging from about 65 to 75, usually from about 67 to 73° C., or about 72° C., and maintained for a period of time ranging from about 15 sec to 20 min, usually from about 30 sec to 5 min.

The above cycles of denaturation, annealing and polymerization may be performed using an automated device, typically known as a thermal cycler. Thermal cyclers that may be employed are described elsewhere herein as well as in U.S. Pat. Nos. 5,612,473; 5,602,756; 5,538,871; and 5,475,610, the disclosures of which are herein incorporated by reference.

The methods of the invention can also be used in non-PCR based applications to detect a target nucleic acid sequence, where such target that may be immobilized on a solid support. Methods of immobilizing a nucleic acid sequence on a solid support are known in the art and are described in Ausubel et al., supra, and in protocols provided by the manufacturers, e.g. for membranes: Pall Corporation, Schleicher & Schuell; for magnetic beads: Dynal; for culture plates: Costar, Nalgenunc; and for other supports useful according to the invention, CPG, Inc.

The person skilled in the art of nucleic acid amplification knows the existence of other rapid amplification procedures such as ligase chain reaction (LCR), transcription-based amplification systems (TAS), self-sustained sequence replication (3SR), nucleic acid sequence-based amplification (NASBA), strand displacement amplification (SDA) and branched DNA (bDNA) (Persing et al, 1993. Diagnostic Molecular Microbiology: Principles and Applications, American Society for Microbiology, Washington, D.C.). The scope of this invention is not limited to the use of amplification by PCR, but rather includes the use of any nucleic acid amplification method or any other procedures which may be useful with the sequences of the invention for the detection and/or quantification of nucleic acid encoding α-amylase.

Further, variations on the exact amounts of the various reagents and on the conditions for the PCR or other suitable amplification procedure (e.g., buffer conditions, cycling times, etc.) that lead to similar amplification or detection/quantification results are known to those of skill in the art and are considered to be equivalents. In one embodiment, the subject QPCR detection has a sensitivity of detecting fewer than 50 copies (preferably fewer than 25 copies, more preferably fewer than 15 copies, still more preferably fewer than 10 copies) of target nucleic acid (e.g., genomic or cDNA) in a sample. In one embodiment, a hot-start PCR reaction is performed (e.g., using a hot start Taq DNA polymerase) so as to improve PCR reaction by decreasing background from non-specific amplification and to increase amplification of the desired extension product.

Controls

The PCR or QPCR reaction of the present invention may contain various controls. Such controls should include a “no template” negative control, in which primers, buffer, enzyme(s) and other necessary reagents (e.g., magnesium chloride, nucleotides) are cycled in the absence of added test sample. A positive control including a known target nucleic acid should also be run in parallel to monitor the efficiency of the amplification/detection reaction and to facilitate quantification of the target nucleic acid. The positive control is ideally an endogenous “housekeeping” gene, such as aldehyde dehydrogenase. Both positive controls and negative controls may be included in the amplification reaction. A single reaction may contain either a positive control, a negative control, or a sample template, or a single reaction may contain both a sample template and a positive control.

In addition to “no template” controls, negative controls can also include amplification reactions with non-specific target nucleic acid included in the reaction, or can be samples prepared using any or all steps of the sample preparation (from nucleic acid extraction to amplification preparation) without the addition of a test sample (e.g., each step uses either no test sample or a sample known to be free of α-amylase).

Confirmation of Primer Extension Product

If nucleic acid encoding α-amylase is present in the test sample, a single amplification product results. This amplification product is about 50 to about 100 base pairs in length, preferably about 82 base pairs in the length, whose termini are defined by the oligonucleotide primer(s) of the present invention (e.g., SEQ ID NO:3 and 4). This polynucleotide sequence (i.e., the amplification product or primer extension product) then serves as a template for the next reaction.

If desired, the identity of the primer extension or amplification product can be confirmed using standard molecular techniques including (for example) a Southern blot assay. In a Southern blot assay, the amplification products are separated by electrophoresis, transferred to a membrane (i.e. nitrocellulose, nylon, etc.), reacted with an oligonucleotide probe or any portion of the nucleic acid sequence specific for the detection of nucleic acid encoding α-amylase. The probe is then modified to enable detection. The modification methods can be the incorporation of a radiolabeled nucleotide or any number of non-radioactive labels (such as biotin). The probe used in the Southern blot assay can be prepared using routine, standard methods. For example, the probe can be isolated, cloned and restricted using routine techniques known in the art or can be made using the chemical synthesis methods described previously herein.

Alternatively, the amplification products can be detected using dot blot analysis. Dot blot analysis involves adhering an oligonucleotide probe (such as the one described herein) to a nitrocellulose or solid support such as, but not limited to, a bead (such as, but not limited to, polystyrene beads, magnetic beads or non-magnetic beads, etc), walls of a reaction tray, strips (such as, but not limited to nitrocellulose strips), or a test tube. The sample containing the labeled amplification product is added, reacted, and washed to removed unbound sample, and a labeled, amplified product attached to the probe is visualized using routine techniques known in the art. A more stringent way to verify the primer extension product or amplification product is through direct sequencing using techniques well known in the art.

Signal Detection

The amount of target nucleic acid can be quantified, for example, according to an increase in detectable fluorescence emitted by a fluorophore (i.e., “signal”). An “increase in fluorescence,” as used herein, refers to an increase in detectable fluorescence emitted by a fluorophore. An increase in fluorescence may result, for example, from cleavage of the hybridized probe from the target nucleic acid. An increase in fluorescence may alternatively result, for example, when the distance between a fluorophore and a quencher is increased, for example due to the spatial separation of the quencher from the fluorophore, such that the quenching is reduced (e.g., due to the hybridization and associated linearization of a stem-loop structured probe, for example, molecular beacon probes).

The sample may be screened for an increase in fluorescence using any convenient means, e.g., a suitable fluorometer, such as a thermostable-cuvette or plate-reader fluorometer. Fluorescence is suitably monitored using a known fluorometer. The signals from these devices, for instance in the form of photo-multiplier voltages, are sent to a data processor board and converted into a spectrum associated with each sample tube. Multiple tubes, for example 96 tubes, can be assessed at the same time. Data may be collected in this way at frequent intervals, for example once every 10 ms throughout the reaction, once per cycle, or once after each of the final cycles, such as after the last 5, 4, 3, or 2 cycles. By monitoring the fluorescence of the reactive molecule from the sample during each cycle, the progress of the amplification reaction can be monitored in various ways. For example, the data provided by melting peaks can be analyzed, for example by calculating the area under the melting peaks and this data plotted against the number of cycles.

Screening the mixture for a change in fluorescence provides one or more assay results, depending on whether the sample is screened once at the end of the primer extension reaction (e.g., traditional PCR methods), or multiple times, e.g., after each cycle, of an amplification reaction (e.g., as is done in real time PCR monitoring).

The data generated as described above can be interpreted in various ways. In its simplest form, an increase in fluorescence from the sample over the course of or at the end of the amplification reaction is indicative of the presence of the target sequence, i.e., primer extension product, suggestive of the fact that the amplification reaction has proceeded and therefore the target sequence was present in the sample.

Quantification is also possible by monitoring the amplification reaction throughout the amplification process. In this manner, a reaction mixture is readily screened for the presence of nucleic acid encoding α-amylase. The methods are suitable for detection and/or quantification of either α-amylase alone, or for multiplex analyses in which two or more different oligonucleotide probes corresponding to α-amylase and one or more additional gene(s) of interest is employed to screen for both (or multiple) genes. In some embodiments, the additional gene of interest is the endogenous control gene. Where multiple genes are screened simultaneously, the type of signaling molecule (e.g., the fluorophore, or fluorophore/quencher combination) used with each primer/probe set should be readily distinguishable in a multiplex assay. A number of convenient fluorophore/quencher pairs are detailed in the literature (for example Glazer, et al. (1997) Current Opinion in Biotechnology 8:94-102) and in catalogues such as those from Molecular Probes and Applied Biosystems.

Kits

Also provided are kits for practicing the subject methods. The kits according to the present invention will comprise at least: (a) a labeled oligonucleotide, where the kit includes two or more distinguishable oligonucleotides, e.g., that hybridize to α-amylase; and (b) instructions for using the provided labeled oligonucleotide(s) in a high fidelity amplification, e.g., PCR, reaction. The kits may separately provide oligonucleotides corresponding to nucleic acid encoding α-amylase, may provide oligonucleotides corresponding to α-amylase and one or more gene(s) of interest packaged together but in separate reaction components, or may provide oligonucleotides corresponding to both (or multiple) genes packaged in the same reaction components.

The subject kits may further comprise additional reagents which are required for or convenient and/or desirable to include in the reaction mixture prepared during the subject methods, where such reagents include: one or more polymerases; an aqueous buffer medium (either prepared or present in its constituent components, where one or more of the components may be premixed or all of the components may be separate), and the like.

The various reagent components of the kits may be present in separate containers, or may all be precombined into a reagent mixture for combination with template nucleic acid.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means for providing instructions may be present in the kits.

### EXPERIMENTAL EXAMPLES

### Example 1

### Detection of Synthetic α-Amylase Gene

Materials and Methods

| | |
|---|---|
| Equipment: | Applied Biosystems 7900HT |
| Taq DNA polymerase: | 2× JUMPSTART mix for QPCR (Sigma- |
| | Aldrich) 550 ul of 1M MgCl₂ and 50 |
| | ul of 600 uM Sulforhodamine (Rox) stock |
| ROX reference dye: | Sulforhodamine 101 (Sigma-Aldrich) |
| PCR Plate: | 384-well barcoded PCR plate (Applied |
| | Biosystems) |
| Optical Plate Film: | TempPlate RT Optical Film (USA Scientific) |

DNA Extraction and Quantification

Quantitative PCR analysis was performed on genomic DNA extracted from corn powder samples using a modified Promega DNA Extraction protocol. DNA quantity was determined by A260 standard methods and verified by ethidium bromide staining after agarose gel electrophoresis, using lambda DNA as a standard. The target nucleic acid comprises a synthetic sequence (SEQ ID NO:1) that has been optimized for maize expression. This synthetic sequence is based on the nucleic acid sequence that encodes the 797GL3 α-amylase gene (SEQ ID NO:2) and is herein referred to as “SynAmylase.” The synthetic sequence is described in U.S. Pat. No. 7,102,057, herein incorporated by reference.

Primer/Probe Stock Preparation

TAQMAN®-compatible probes are obtained Invitrogen Corporation (Carlsbad, Calif.) wherein the fluorophore FAM is attached to the 5′ terminus of the probe sequence for the α-amylase gene (SEQ ID NO:5) and the quencher TAMRA is attached to the 3′ terminus. The endogenous control gene (EC) probe is obtained in the same manner, except with the addition of TET to the 5′ terminus instead of FAM. The primers for the endogenous control gene are set forth in SEQ ID NO:6 and 7, and the probe sequence for the endogenous control gene is set forth in SEQ ID NO:8. See Table 1.

TABLE 1

| | | |
|---|---|---|
| | | SEQ ID |
| Name | Sequence | NO: |

| | | | |
|---|---|---|---|
| SynAmylase | | | |
| Sense primer | synAmylase - F | CAAGCAGGAGCTCATCAACATG | 3 |
| Antisense primer | synAmylase - R | GCCCTGTGGTTGATCACGAT | 4 |
| Probe | synAmylase - P | TCCGCGATGACCTTGATGCCGTA | 5 |
| | FAM | | |
| endogenous | | | |
| control | | | |
| Sense primerm | ZmADH-267F | GAACGTGTGTTGGGTTTGCAT | 6 |
| Antisense primer | ZmADH-337R | TCCAGCAATCCTTGCACCTT | 7 |
| Probe | ZmADHtet-316R | TGCAGCCTAACCATGCGCAGGGTA | 8 |

Sense and antisense primer (SEQ ID NO:3 and 4) stock solutions are prepared by suspending lyophilized primers in Tris-EDTA (TE) buffer at 900 nM. These stock solutions are stored at −20° C. Probe stock solutions are prepared by suspending lyophilized oligonucleotide in TE at 100 uM. These stock solutions are stored at −20° C. in the dark.

Primer master mixes are prepared for both the synthetic α-amylase gene (SynAmylase) and the endogenous control gene. A 50× concentration of each of the primer master mixes is prepared as follows:

TABLE 2
50× SynAmylase primer/probe stock

| | | | Final concentration |
|---|---|---|---|
| | Name | 50× | (1×) |
| Forward primer | SynAmylase-F | 4.5 uM | 900 nM |
| Reverse primer | SynAmylase-R | 4.5 uM | 900 nM |
| Probe | SynAmylase-P | 0.5 uM | 100 nM |
| | FAM | | |

TABLE 3
50× EC primer/probe stock

| | | | Final concentration |
|---|---|---|---|
| | Name | 50× | (1×) |
| Forward primer | ZmADH-267 | 4.5 uM | 900 nM |
| Reverse primer | ZmADH-337R | 4.5 uM | 900 nM |
| Probe | AmADHtet-316R | 0.5 uM | 100 nM |

PCR Set-Up and Reaction Conditions

PCR reactions were set up in which each reaction contained 5 ul of 2× JUMPSTART™ mix with supplements, 0.2 ul of either (singleplex reaction) or both (multiplex reaction) of SynAmylase 50× primer/probe mix and 50× EC primer/probe mix, 3 ul genomic DNA, and nuclease-free water to bring the total volume to 10 ul per reaction. The cycling conditions are outlined in Table 4.

TABLE 4
PCR Cycling conditions

| Cycle No. | Temperature (° C.) | Time | |
|---|---|---|---|
| 1 | 95 | 10 | minutes |
| 40 | 95 | 10 | seconds |
| | 60 | 1 | minute |

Assay Dynamic Range and Amplification Efficiency

Both SynAmylase and EC were tested using genomic DNA from mixed corn powder (transgenic corn powder (3272×Bt11) premixed with non-transgenic corn powder at different ratios). The extracted DNA was diluted in a wide dynamic range including 50, 10, 4, 2, 1, 0.5, and 0.25 ng/ul. Both singleplex (i.e., SynAmylase and EC in separate reaction tubes) and duplex (both in the same tube) reactions were tested. The amplification slopes, R squares (RSQ), and efficiencies (E) are provided in Table 5.

TABLE 5

| | | |
|---|---|---|
| Slope | RSQ | E* |

SINGLEPLEX REACTION

| | | | |
|---|---|---|---|
| FAM | −3.44 | 1.0 | 0.95 |
| TET | −3.40 | 1.0 | 0.97 |
| Difference | −0.04 | | −0.01 |

DUPLEX REACTION

| | | | |
|---|---|---|---|
| FAM | −3.19 | 1.0 | 1.06 |
| TET | −3.26 | 1.0 | 1.03 |
| Difference | 0.07 | | 0.03 |

DIFFERENCE BETWEEN SINGLEPLEX AND DUPLEX

| | | |
|---|---|---|
| FAM | −0.25 | −0.1 |
| TET | −0.14 | −0.06 |
| Difference | −0.11 | −0.05 |

*E = 1 − \hat{ }(1/slope) − 1

As shown in Table 5, for all tested DNA concentrations, both assays worked properly as the assay efficiencies for SynAmylase (FAM) and the EC (TET) were higher than 0.95. The differences in the efficiency of TET and FAM between singleplex and duplex reactions are no more than 0.1.

Limit of Detection (LOD) Test

The LOD of SynAmylase was tested by singleplex and duplex reactions using the genomic DNA extracted from mixed corn powder. The mixed corn powder was prepared by mixing transgenic corn powder (3272×Bt11) with non-transgenic corn powder in a series range of 1, 0.5, 0.25, 0.1, 0.05, and 0%. Sixteen replicates were performed for each reaction using extracted DNA. The Ct values and their standard deviation (STDEV), as well as the efficiencies of the amplification were calculated. The suggested criteria for Ct value are 4 less than that of the non-genetically modified sample (wildtype, or “wt”) and the CV % was less than 3%.

TABLE 6
CT and CV % of FAM (SynAmylase) in singleplex reaction:

| GM % | Mean | STDEV | CV % | Diff to wt |
|---|---|---|---|---|
| 1 | 29.81 | 0.24 | 0.8 | 9.96 |
| 0.50 | 30.32 | 0.22 | 0.7 | 9.45 |
| 0.25 | 31.11 | 0.33 | 1.1 | 8.67 |
| 0.10 | 32.34 | 0.37 | 1.1 | 7.43 |
| 0.05 | 32.88 | 0.45 | 1.4 | 6.89 |
| 0% (i.e., 100% wt) | 39.77 | 0.91 | 2.3 | |

TABLE 7
CT and CV % of FAM (SynAmylase) in duplex reaction:

| GM % | Mean | STDEV | CV % | Diff to wt |
|---|---|---|---|---|
| 1 | 28.4 | 0.37 | 1.3 | 11.6 |
| 0.50 | 29.27 | 0.33 | 1.1 | 10.73 |
| 0.25 | 30.61 | 0.66 | 2.2 | 9.39 |
| 0.10 | 32.19 | 0.86 | 2.7 | 7.81 |
| 0.05 | 34.16 | 2.08 | 6.1 | 5.84 |
| 0% (i.e., 100% wt) | 40.00 | 0.00 | 0.0 | |

Based on these results, the LOD of SynAmylase in singleplex and duplex reaction are 0.05% and 0.1%, respectively.

### Example 2

### Detection of Phosphomannose Isomerase Gene (PMI)

Materials and Methods

| | |
|---|---|
| Equipment: | Applied Biosystems 7900HT |
| Taq DNA polymerase: | 2× JUMPSTART mix for QPCR (Sigma- |
| | Aldrich) 550 ul of 1M MgCl₂ and 100 |
| | ul of 300 uM Sulforhodamine (Rox) stock |
| ROX reference dye: | Sulforhodamine 101 (Sigma-Aldrich) |
| PCR Plate: | 384-well barcoded PCR plate (Applied |
| | Biosystems) |
| Optical Plate Film: | TempPlate RT Optical Film (USA Scientific) |

DNA Extraction and Quantification

Quantitative PCR analysis was performed on genomic DNA extracted from corn powder samples using a modified Promega DNA Extraction protocol. DNA quantity was determined by A260 standard methods and verified by ethidium bromide staining after agarose gel electrophoresis, using lambda DNA as a standard. The target nucleic acid is the phosphomannose isomerase gene described in U.S. Pat. No. 6,858,777, herein incorporated by reference in its entirety.

Primer/Probe Stock Preparation

TAQMAN®-compatible probes are obtained Invitrogen Corporation (Carlsbad, Calif.) wherein the fluorophore FAM is attached to the 5′ terminus of the probe sequence for the PMI gene (SEQ ID NO:11) and the quencher TAMRA is attached to the 3′ terminus. The endogenous control gene (EC) probe is obtained in the same manner as described in Example 1. See Table 8.

TABLE 8

| | | | SEQ ID |
|---|---|---|---|
| PMI | Name | Sequence | NO: |
| Sense | PMI-1015F | CCGGGTGAATCAGCGTTT | 9 |
| primer | | | |
| Antisense | PMI-1074R | GCCGTGGCCTTTGACAGT | 10 |
| primer | | | |
| Probe | PMI-1035F | TGCCGCCAACGAATCACCGG | 11 |

Sense and antisense primer (SEQ ID NO:9 and 10) stock solutions are prepared by suspending lyophilized primers in Tris-EDTA (TE) buffer at 900 nM. These stock solutions are stored at −20° C. Probe stock solutions are prepared by suspending lyophilized oligonucleotide in TE at 100 uM. These stock solutions are stored at −20° C. in the dark.

Primer master mixes are prepared for both the PMI gene (SynAmylase) and the endogenous control gene. A 50× concentration of each of the primer master mixes is prepared as follows:

TABLE 9
50× PMI primer/probe stock

| | | | Final concentration |
|---|---|---|---|
| | Name | 50× | (1×) |
| Forward primer | PMI-1015F | 4.5 uM | 900 nM |
| Reverse primer | PMI-1074R | 4.5 uM | 900 nM |
| Probe | PMI-1035F | 0.5 uM | 100 nM |

PCR Set-Up and Reaction Conditions

PCR reactions were set up in which each reaction contained 5 ul of 2× JUMPSTART™ mix with supplements, 0.2 ul of either (singleplex reaction) or both (multiplex reaction) of PMI 50× primer/probe mix and 50× EC primer/probe mix, 3 ul genomic DNA, and nuclease-free water to bring the total volume to 10 ul per reaction. The cycling conditions are outlined in Table 10.

TABLE 10
PCR Cycling conditions

| Cycle No. | Temperature (° C.) | Time | |
|---|---|---|---|
| 1 | 95 | 10 | minutes |
| 40 | 95 | 15 | seconds |
| | 60 | 1 | minute |

Assay Dynamic Range and Amplification Efficiency

Both PMI and EC were tested using genomic DNA from mixed corn powder (transgenic corn powder (3272×Bt11) premixed with non-transgenic corn powder at different ratios). The extracted DNA was diluted in a wide dynamic range from 0.25-50 ng/ul. Both singleplex (i.e., PMI and EC in separate reaction tubes) and duplex (both in the same tube) reactions were tested. The amplification slopes, R squares (RSQ), and efficiencies (E) are provided in Table 11.

TABLE 11

| | | |
|---|---|---|
| Slope | RSQ | E* |

SINGLEPLEX REACTION

| | | | |
|---|---|---|---|
| FAM | −3.45 | 1.0 | 0.95 |
| TET | −3.46 | 1.0 | 0.95 |
| Difference | 0.01 | | 0.00 |

DUPLEX REACTION

| | | | |
|---|---|---|---|
| FAM | −3.36 | 1.0 | 0.99 |
| TET | −3.33 | 1.0 | 1.00 |
| Difference | −0.02 | | −0.01 |

DIFFERENCE BETWEEN SINGLEPLEX AND DUPLEX

| | | |
|---|---|---|
| FAM | −0.09 | −0.04 |
| TET | −0.13 | −0.05 |
| Difference | 0.03 | 0.01 |

*E = 1 − \hat{ }(1/slope) − 1

As shown in Table 11, for all tested DNA concentrations, both assays worked properly as the assay efficiencies for PMI (FAM) and the EC (TET) were higher than 0.95. The differences in the efficiency of TET and FAM between singleplex and duplex reactions are less than 0.1.

Limit of Detection (LOD) Test

The LOD of PMI was tested by singleplex and duplex reactions using the genomic DNA containing PMI diluted in a series range of 1.0, 0.5, 0.25, 0.1, 0.05, 0% in 10 ng/ul of the wild type genomic DNA. Sixteen replicates were performed for each reaction using extracted DNA. The Ct values and their standard deviation (STDEV), as well as the efficiencies of the amplification were calculated. The suggested criteria for Ct value (from the lowest amount of genomic DNA in which PMI is significantly amplified) is 4 cycles less than that of the non-genetically modified sample (wildtype, or “wt”) and the CV % was less than 3%.

TABLE 12
CT and CV % of FAM (PMI) in singleplex reaction:

| GM % | Mean | STDEV | CV % | Diff to wt |
|---|---|---|---|---|
| 1 | 28.64 | 0.32 | 1.1 | 9.59 |
| 0.50 | 29.45 | 0.34 | 1.2 | 8.79 |
| 0.25 | 30.30 | 0.38 | 1.3 | 7.93 |
| 0.10 | 31.39 | 0.41 | 1.3 | 6.84 |
| 0.05 | 31.95 | 0.44 | 1.4 | 6.29 |
| 0% (i.e., 100% wt) | 38.23 | 1.92 | 5.0 | |

TABLE 13
CT and CV % of FAM (PMI) in duplex reaction:

| GM % | Mean | STDEV | CV % | Diff to wt |
|---|---|---|---|---|
| 1 | 28.26 | 0.29 | 1.0 | 11.74 |
| 0.50 | 29.13 | 0.15 | 0.5 | 10.87 |
| 0.25 | 30.81 | 0.58 | 1.9 | 9.19 |
| 0.10 | 32.4 | 0.85 | 2.6 | 7.60 |
| 0.05 | 34.14 | 1.42 | 4.2 | 5.86 |
| 0% (i.e., 100% wt) | 40.00 | 0.00 | 0.0 | |

Based on these results, the LOD of PMI in singleplex and duplex reaction are 0.05% and 0.1%, respectively.

That which is claimed:

1. A composition for the detection of a nucleic acid encoding α-amylase in a sample, wherein said composition comprises an oligonucleotide comprising SEQ ID NO:3 and an oligonucleotide comprising SEQ ID NO:4.

2. The composition of claim 1, wherein said nucleic acid encoding α-amylase comprises nucleotides 258-341 of SEQ ID NO:1, or a complement thereof.

3. The composition of claim 1, further comprising an oligonucleotide comprising SEQ ID NO:5, or a complement thereof.

4. The composition of claim 3, wherein said oligonucleotide comprising SEQ ID NO:5 further comprises a fluorophore and a quencher molecule.

5. The composition of claim 3, wherein said oligonucleotide comprising SEQ ID NO:5 further comprises a phosphate group attached to the 3′ terminus of said oligonucleotide.

6. A kit for the detection of a nucleic acid encoding α-amylase in a sample comprising an oligonucleotide comprising SEQ ID NO:3 and an oligonucleotide comprising SEQ ID NO:4.

7. The composition of claim 6, wherein said nucleic acid encoding α-amylase comprises nucleotides 258-341 of SEQ ID NO:1, or a complement thereof.

8. The composition of claim 6, further comprising an oligonucleotide comprising SEQ ID NO:5, or a complement thereof.

9. The composition of claim 8, wherein said oligonucleotide comprising SEQ ID NO:5 further comprises a fluorophore and a quencher molecule.

10. The composition of claim 8, wherein said oligonucleotide comprising SEQ ID NO:5 further comprises a phosphate group attached to the 3′ terminus of said oligonucleotide.

11. A method for detecting the presence of a nucleic acid encoding α-amylase in a sample using polymerase-based amplification of a target nucleic acid region present in said α-amylase, said method comprising:
a) providing a test sample suspected of containing a nucleic acid encoding α-amylase;
b) contacting said sample with at least a first and a second oligonucleotide primer under conditions sufficient to provide polymerase-based nucleic acid amplification products comprising the target region, wherein said at least a first oligonucleotide primer comprises SEQ ID NO:3 and said at least a second oligonucleotide primer comprises SEQ ID NO:4; and,
c) detecting the amplified products.

12. The composition of claim 11, wherein said nucleic acid encoding α-amylase comprises nucleotides 258-341 of SEQ ID NO:1, or a complement thereof.

13. The composition of claim 11, further comprising an oligonucleotide comprising SEQ ID NO:5, or a complement thereof.

14. The composition of claim 13, wherein said oligonucleotide comprising SEQ ID NO:5 further comprises a fluorophore and a quencher molecule.

15. The composition of claim 13, wherein said oligonucleotide comprising SEQ ID NO:5 further comprises a phosphate group attached to the 3′ terminus of said oligonucleotide.

16. The method of embodiment 13, wherein said polymerase-based amplification is quantitative polymerase chain reaction (QPCR).
